(19) United States
(12) Patent Application Publication
(10) Pub. No.: US 2023/0201157 A1
(43) Pub. Date: Jun. 29, 2023

(54) COMPOSITIONS OF CANNABINOIDS AND METHODS OF USING SAME

(71) Applicant: ASANA BIO GROUP LTD., Tel Aviv (IL)

(72) Inventor: Sari Prutchi SAGIV, Kibbutz HaMaapil (IL)

(73) Assignee: ASANA BIO GROUP LTD., Tel Aviv (IL)

(21) Appl. No.: 17/927,073

(22) PCT Filed: May 24, 2021
(86) PCT No.: PCT/IL2021/050605

§ 371 Date: Nov. 22, 2022

Related U.S. Application Data
(60) Provisional application No. 63/029,504, filed on May 24, 2020.

(51) Int. Cl. A61K 31/352; A61K 31/05; A61K 45/06; A61K 31/192

(52) U.S. Cl. CPC A61K 31/352; A61K 31/05; A61K 31/192; A61K 45/06

(57) ABSTRACT

The present invention is directed to a composition including a cannabinoid and its acid precursor, and optionally further includes a terpene and/or a flavonoid. Further provided are methods of using the composition, such as for inhibiting IL-6 secretion, and for treating a subject afflicted with an IL-6-related disease, a COX-related disease, or both.

### CROSS-REFERENCE TO RELATED APPLICATION

This application claims the benefit of priority to U.S. Provisional Pat. Application No. 63/029,504, titled “COMPOSITIONS OF CANNABINOIDS AND METHODS OF USING SAME”, filed May 24, 2020, the contents of which are incorporated herein by reference in their entirety.

### FIELD OF THE INVENTION

The present invention relates to compositions of cannabinoids, and methods of using same, such as for inhibiting IL-6 secretion, reducing cyclooxygenase expression, and for treatment or prevention of a disease, e.g., an inflammatory disease.

### BACKGROUND

The cannabis plant (Cannabis saliva) has been in use for medical purposes for thousands of years. Medical Cannabis is nowadays prescribed for prevention of nausea and vomiting associated with cancer chemotherapy, and for the treatment of anorexia associated with AIDS and cancer.

Cannabis plants produce a group of natural chemicals called Cannabinoids, among them Δ9-tetrahydrocannabinol (THC), Cannabidiol and ajulemic acid.

The immune-modulatory and anti-inflammatory properties of cannabinoids, for example Cannabidiol, have been shown in animal models of various inflammatory diseases including multiple sclerosis, diabetes mellitus, inflammatory bowel disease and rheumatoid arthritis, CBD mediates its anti-inflammatory effects by suppressing T cell proliferation, by shifting the balance from TH1 to Th2 cytokines, inhibiting the pro-inflammatory cytokine release including INFy, TNFα, IL-1β, IL-6, IL-17 and stimulating the anti-inflammatory cytokine release including IL-4, IL-5, IL-10, IL-13.

The use of a cannabinoid, e.g., cannabidiol, for treating inflammatory diseases such as rheumatoid arthritis, multiple sclerosis and Crohn’s Disease, and medicinal preparations containing CBD for use in treating such diseases, has been described.

Pharmaceutical compositions comprising cannabinoids, e.g., cannabidiol derivatives, which have analgesic, antianxiety, anticonvulsive, neuroprotective, antipsychotic anticancer activity, as well as beneficiary effects on women’s health, have been described.

Although the prior art teaches uses of cannabinoids, e.g., CBD, THC, etc., for treating various types of disease, e.g., inflammatory diseases, it does not describe or suggest the use of specific combinations and/or mixtures of a cannabinoid(s) and its/their respective precursor acid(s), for such treatments.

### SUMMARY

According to a first aspect, there is provided a composition comprising: cannabidiol (CBD), cannabidiolic acid (CBDA), tetrahydrocannabinol (THC), and tetrahydrocannabinol acid (THCA), wherein: i. the weight per weight or molar ratio of CBD to CBDA is in the range of 1:2 to 2:1, ii. the w/w (or molar) ratio of THC to THCA is in the range of 1:1 to 4:1, and iii. the w/w (or molar) ratio of CBD and CBDA to THC and THCA is in the range of 1:2 to 2:1.

According to another aspect, there is provided a composition consisting essentially of CBD, CBDA, THC, THCA, CBG, apigenin, BCP, and β myrcene.

According to another aspect, there is provided a composition comprising CBD and CBDA in a w/w or molar ratio ranging from 1:2 to 2:1.

According to another aspect, there is provided a composition consisting essentially of CBD, CBDA, CBG, CBN, THCV, apigenin, BCP, and β myrcene.

According to another aspect, there is provided a method for reducing or inhibiting IL-6 secretion by an immune cell, comprising contacting the immune cell with an effective amount of the composition of the invention, thereby reducing or inhibiting IL-6 secretion by the immune cell.

According to another aspect, there is provided a method for treating a subject afflicted with an IL-6-related disease, COX-related disease, or both, comprising the step of administering to the subject a therapeutically effective amount of the composition of the invention, thereby treating the subject afflicted with an IL-6-related disease, COX-related disease, or both.

In some embodiments, the w/w or molar ratio of THC to THCA is in the range of 2:1 to 2.5:1.

In some embodiments, the w/w or molar ratio of CBD to CBDA is 1:1.

In some embodiments, the w/w or molar ratio of CBD and CBDA to THC and THCA is 1:1.

In some embodiments, the composition further comprises an additional compound selected from the group consisting of: cannabigerol (CBG), cannabinol (CBN), tetrahydrocannabivarin (THCV), apigenin, beta caryophyllene (BCP), β myrcene, and any combination thereof.

In some embodiments, the w/w or molar ratio of: (a) the combination of CBD and CBDA, (b) the combination of THC and THCA, or (c) the combination of (a) and (b), to the additional compound is in the range of 1:1 to 1:6.

In some embodiments, the molar ratio of: (a) the combination of CBD and CBDA, (b) the combination of THC and THCA, or (c) the combination of (a) and (b) to any one of: CBG, apigenin, BCP, and β myrcene, is in the range of 1:1 to 1:10.

In some embodiments, the w/w or molar ratio of CBD to CBDA is 1:1.

In some embodiments, the composition further comprises an additional compound selected from the group consisting of: CBG, CBN, THCV, apigenin, BCP, β myrcene, and any combination thereof.

In some embodiments, the w/w or molar ratio of CBD and CBDA, to the additional compound is in the range of 1:1 to 1:6.

In some embodiments, the molar ratio of the combination of CBD and CBDA to any one of: CBG, CBN, THCV, apigenin, BCP, and β myrcene, is in the range of 1:1 to 1:10.

In some embodiments, the composition comprises a total concentration of cannabinoids ranging from 15 to 50 µM.

In some embodiments, the composition further comprises a pharmaceutically acceptable carrier.

In some embodiments, the composition is for use in the treatment of an interleukin-6 (IL-6)-related disease, a cyclooxygenase (COX)-related disease, or both.

In some embodiments, the disease is selected from the group consisting of: an immune disease, an inflammatory disease, endometriosis, dysmenorrhea, and dyspareunia.

In some embodiments, the inflammatory disease is an inflammatory lung disease, or a viral induced inflammation.

In some embodiments, the immune cell comprises a monocyte, a macrophage, or a combination thereof.

In some embodiments, contacting is contacting in-vivo, ex-vivo or in-vitro.

In some embodiments, the method further comprises a step of determining the level of secreted IL-6, COX expression level, or both, of the subject, wherein any one of : (a) a secretion level of IL-6, (b) an expression level of COX , or a combination of (a) and (b), above a predetermined threshold indicates the subject is suitable for treatment with the composition, and wherein the determining step is performed before the administering step.

In some embodiments, administering is topically administering, orally administering, or both.

In some embodiments, treating comprises one or more of: increasing immune cell viability, reducing nitric oxide production, reducing COX expression, reducing IL-6 secretion, or any combination thereof, in the subject.

In some embodiments, COX is COX1, COX2, or both.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

In addition to the exemplary aspects and embodiments described above, further aspects and embodiments will become apparent by reference to the study of the following detailed description.

### BRIEF DESCRIPTION OF THE FIGURES

FIG. 1 includes vertical bar graphs showing the effect of different concentrations (50, 25, and 12.5 µM) of various cannabinoids on cell viability (upper graph), nitric oxide production level (middle graph), and a ratio of cell viability/nitric oxide production level (lower graph). THC (tetrahydrocannabinol); THCA (tetrahydrocannabinolic acid); CBD (cannabidiol); CBDA (cannabidiolic acid); THCV (tetrahydrocannabivarin); THCVA (tetrahydrocannabivarinic acid); CBG (cannabigerol); CBGA (cannabigerolic acid); CBN (cannabinol); CBNA (cannabinolic acid); CBC (cannabichromene); and CBCA (cannabichromenic acid).

FIG. 2 includes vertical bar graphs showing the effect of β myrcene, beta caryophyllene (BCP), and apigenin on cell viability (upper graph), nitric oxide production level (middle graph), and a ratio of cell viability/nitric oxide production level (lower graph).

FIG. 3 includes vertical bar graphs showing the effect of different concentrations (50, 25, and 12.5 µM) of THC and THCA combinations (1:1 molar ratio, 4:1 molar ratio, 1.5:1 molar ratio, and 1:4 molar ratio) on cell viability (upper graph), nitric oxide production level (middle graph), and a ratio of cell viability/nitric oxide production level (lower graph).

FIG. 4 includes vertical bar graphs showing the effect of combinations 1-3 on cell viability (upper graph), nitric oxide production level (middle graph), and a ratio of cell viability/nitric oxide production level (lower graph). Combination 1: 40 µM comprising: THC:THCA - 3.5 µM : 1.5 µM (70:30), CBD:CBDA - 2.5 µM : 2.5 µM (50:50), CBG-5 µM, CBN - 5 µM, THCV - 5 µM, Apigenin - 5 µM, BCP - 5 µM, and β myrcene - 5 µM; Combination 2: 70 µM comprising: CBD:CBDA - 5 µM : 5 µM (50:50), CBG - 10 µM, CBN - 10 µM, THCV - 10 µM, Apigenin - 10 µM, BCP - 10 µM, and β myrcene - 10 µM; and Combination 3: 60 µM comprising: THC:THCA - 7 µM : 3 µM (70:30), CBD:CBDA - 5 µM : 5 µM (50:50), CBG - 10 µM, Apigenin - 10 µM, BCP - 10 µM, and β myrcene -10 µM.

FIG. 5 includes vertical bar graphs showing the effect of combinations 4-5 on cell viability (upper graph), nitric oxide production level (middle graph), and a ratio of cell viability/nitric oxide production level (lower graph). Combination 4: 50 µM comprising: THC:THCA - 7 µM : 3 µM (70:30), CBD:CBDA - 5 µM : 5 µM (50:50), CBG - 10 µM, Apigenin - 10 µM, and BCP - 10 µM;and Combination 5: 50 µM comprising: CBD:CBDA - 5 µM : 5 µM (50:50), CBG - 10 µM, Apigenin - 10 µM, BCP - 10 µM, and β myrcene -10 µM.

FIG. 6 includes a vertical bar graph showing the effect of CBD (12.5 µM), CBDA (50 µM), THCV (50 µM), CBG (50 µM), CBGA (12.5 µM), CBN (12.5 µM), or CBCA (50 µM), on the expression levels of cyclooxygenase 1 (COX1) and COX2 as tested in activated macrophages. Negative control included naive cells without treatment or activation.

FIG. 7 includes a vertical bar graph showing the effect of THC (25 µM), THCA (50 µM), THC and THCA (70 µM : 30 µM), THC and THCA (40 µM: 60 µM, THC and THCA (30 µM : 70 µM), THC and THCA (10 µM: 90 µM), on the expression levels of COX1 and COX2. Negative control included naive cells without treatment or activation.

FIG. 8 includes a vertical bar graph showing the effect of β myrcene (50 µM), beta caryophyllene (BCP; 50 µM), and apigenin (25 µM) on the expression levels of COX1 and COX2. Negative control included naive cells without treatment or activation.

FIG. 9 includes a vertical bar graph showing the effect of combinations 1-3 (of FIG. 4) and 4-5 (of FIG. 5) on the expression levels of COX1 and COX2.

FIG. 10 includes a vertical bar graph showing the effect of 10 µM of any one of: THCA, THC, CBDA, CBD, and CBG, 50 µM of any one of: CBN, CBC, and THCV, 60 µM of terpenes, Combinations 1-5 (as in FIGS. 4 and 5), and combinations 1a, 2a , and 3a., on interleukin-6 secretion. Combinations 2-3 which were found to have positive effects, e.g., cell viability/nitric oxide production ratio, and COX expression levels (e.g., FIGS. 4 and 9), were also found to reduce IL-6 secretion compared to control. Combination 1a: 310 µM comprising: CBD:CBDA - 50 µM : 50 µM (50:50), CBN - 10 µM, THCV - 50 µM, Apigenin - 50 µM, BCP - 50 µM, and β myrcene - 50 µM;Combination 2a: 260 µM comprising: CBDA - 50 µM, CBG - 50 µM, CBNA - 10 µM, THCVA - 50 µM, BCP - 50 µM, and β myrcene - 50 µM ; and Combination 3a: 180 µM comprising: CBD - 50 µM, CBC - 10 µM,CBG- 10 µM,CBN- 10 µM,THCV - 10 µM,and BCP - 50 µM.

### DETAILED DESCRIPTION

According to some embodiments, there is provided a composition comprising a cannabinoid and its acid precursor. In some embodiments, the composition further comprises a terpene, a flavonoid, or a combination thereof.

The present invention is based, in part, on the surprising findings that particular combinations of cannabinoids and their respective acid precursors, e.g., cannabidiol (CBD) and cannabidiolic acid (CBDA), and tetrahydrocannabinol (THC) and tetrahydrocannabinol acid (THCA), modulated immune cell activity, including: increased cell proliferation, reduced nitric oxide (NO) production, reduced cyclooxygenase (COX) expression, reduced interleukin-6 (IL-6) secretion, or a combination thereof.

### Composition

In some embodiments, there is provided a composition comprising CBD and CBDA. In some embodiments, there is provided a composition comprising CBD, CBDA, THC, and THCA.

In some embodiments, the weight per weight (w/w) or molar (molar/molar) ratio of CBD to CBDA within a composition as described herein is in the range of 1:10 to 10:1, 1:9 to 9:1, 1:8 to 8:1, 1:7 to 7:1, 1:6 to 6:1, 1:5 to 5:1, 1:4 to 4:1, 1:3 to 3:1, or 1:2 to 2:1. Each possibility represents a separate embodiment of the invention.

In some embodiments, the weight per weight (w/w) or molar (molar/molar) ratio of CBD to CBDA within a composition as described herein is in the range of 1:2 to 2:1. In some embodiments, the w/w or molar/molar ratio of CBD to CBDA within a composition as described herein is 1:1.

In some embodiments, the w/w or molar/molar ratio of THC to THCA within a composition as described herein is in the range of 1:1 to 15:1, 1:1 to 14:1, 1:1 to 13:1, 1:1 to 12:1, 1:1 to 11:1, 1:1 to 10:1, 1:1 to 9:1, 1:1 to 8:1, 1:1 to 7:1, 1:1 to 6:1, 1:1 to 5:1, 1:1 to 4:1, 1:1 to 3:1, or 1:1 to 2:1. Each possibility represents a separate embodiment of the invention. In some embodiments, the w/w or molar/molar ratio of THC to THCA within a composition as described herein is in the range of 1:1 to 4:1, 1:1 to 3:1, or 1:1 to 2:1. In some embodiments, the w/w or molar/molar ratio of THC to THCA within a composition as described herein is in the range of 2:1 to 2.5:1. In some embodiments, the w/w or molar/molar ratio of THC to THCA within a composition as described herein is 7:3.

In some embodiments, the w/w or molar/molar ratio of CBD and CBDA to THC and THCA within a composition as described herein is in the range of 1:8 to 8:1, 1:7 to 7:1, 1:6 to 6:1, 1:5 to 5:1, 1:4 to 4:1, 1:3 to 3:1, or 1:2 to 2:1. Each possibility represents a separate embodiment of the invention. In some embodiments, the w/w or molar/molar ratio of CBD and CBDA to THC and THCA within a composition as described herein is in the range of 1:2 to 2:1. In some embodiments, the w/w or molar/molar ratio of CBD and CBDA to THC and THCA within a composition as described herein is 1:1.

In some embodiments, the composition further comprises an additional compound selected from: cannabigerol (CBG), cannabinol (CBN), tetrahydrocannabivarin (THCV), apigenin, beta caryophyllene (BCP), β myrcene, or any combination thereof.

In some embodiments, the w/w or molar/molar ratio of CBD and CBDA, THC and THCA, or both, to the additional compound within a composition as described herein is in the range of 1:1 to 1:10, 1:1 to 1:9, 1:1 to 1:8, 1:1 to 1:7, 1:1 to 1:6, 1:1 to 1:5, 1:1 to 1:4, 1:1 to 1:3, 1:1 to 1:2, or is 1:1. Each possibility represents a separate embodiment of the invention. In some embodiments, the w/w or molar/molar ratio of CBD and CBDA, THC and THCA, or both, to the additional compound within a composition as described herein is in the range of 1:1 to 1:6.

In some embodiments, a composition comprising: (i) CBD and CBDA, THC, and THCA or (ii) CBD and CBDA, further comprises CBG. In some embodiments, the w/w or molar/molar ratio of (i) or (ii) to CBG within a composition as described herein is in the range of 1:1 to 1:10, 1:1 to 1:8, 1:1 to 1:6, 1:1 to 1:4, 1:1 to 1:3, 1:1 to 1:2, or is 1:1. Each possibility represents a separate embodiment of the invention.

In some embodiments, a composition comprising: (i) CBD and CBDA, THC, and THCA or (ii) CBD and CBDA, further comprises CBN. In some embodiments, the w/w or molar/molar ratio of (i) or (ii) to CBN within a composition as described herein is in the range of 1:1 to 1:10, 1:1 to 1:8, 1:1 to 1:6, 1:1 to 1:4, 1:1 to 1:3, 1:1 to 1:2, or is 1:1. Each possibility represents a separate embodiment of the invention.

In some embodiments, a composition comprising: (i) CBD and CBDA, THC, and THCA or (ii) CBD and CBDA, further comprises THCV. In some embodiments, the w/w or molar/molar ratio of (i) or (ii) to THCV within a composition as described herein is in the range of 1:1 to 1:10, 1:1 to 1:8, 1:1 to 1:6, 1:1 to 1:4, 1:1 to 1:3, 1:1 to 1:2, or is 1:1. Each possibility represents a separate embodiment of the invention.

In some embodiments, a composition comprising: (i) CBD and CBDA, THC, and THCA or (ii) CBD and CBDA, further comprises apigenin. In some embodiments, the w/w or molar/molar ratio of (i) or (ii) to apigenin within a composition as described herein is in the range of 1:1 to 1:10, 1:1 to 1:8, 1:1 to 1:6, 1:1 to 1:4, 1:1 to 1:3, 1:1 to 1:2, or is 1:1. Each possibility represents a separate embodiment of the invention.

In some embodiments, a composition comprising: (i) CBD and CBDA, THC, and THCA or (ii) CBD and CBDA, further comprises BCP. In some embodiments, the w/w or molar/molar ratio of (i) or (ii) to BCP within a composition as described herein is in the range of 1:1 to 1:10, 1:1 to 1:8, 1:1 to 1:6, 1:1 to 1:4, 1:1 to 1:3, 1:1 to 1:2, or is 1:1. Each possibility represents a separate embodiment of the invention.

In some embodiments, a composition comprising: (i) CBD and CBDA, THC, and THCA or (ii) CBD and CBDA, further comprises β myrcene. In some embodiments, the w/w or molar/molar ratio of (i) or (ii) to β myrcene is in the range of 1:1 to 1:10, 1:1 to 1:8, 1:1 to 1:6, 1:1 to 1:4, 1:1 to 1:3, 1:1 to 1:2, or is 1:1. Each possibility represents a separate embodiment of the invention.

In some embodiments, there is provided a composition consisting essentially of CBD, CBDA, THC, THCA, CBG, apigenin, BCP, and β myrcene. In some embodiments, there is provided a composition consisting essentially of CBD, CBDA, CBG, CBN, THCV, apigenin, BCP, and β myrcene.

In some embodiments, the molar ratio of: (i) CBD, CBDA, THC and THCA or (ii) CBD and CBDA, to any one of: CBG, CBN, THCV, apigenin, BCP, and β myrcene, or a combination thereof, is in the range of 1:1 to 1:10, 1:1 to 1:9, 1:1 to 1:8, 1:1 to 1:7, 1:1 to 1:6, 1:1 to 1:5, 1:1 to 1:4, 1:1 to 1:3, 1:1 to 1:2, or is 1:1.

The term “consisting essentially of” denotes that a given compound or substance constitutes the vast majority of the active ingredient’s portion or fraction of the composition.

In some embodiments, consisting essentially of means that the combination of CBD, CBDA, THC, THCA, CBG, apigenin, BCP, and β myrcene constitute at least 95%, at least 98%, at least 99%, or at least 99.9% by weight, of the active ingredient(s) of the composition, or any value and range therebetween. Each possibility represents a separate embodiment of the invention.

In some embodiments, consisting essentially of means that the combination of CBD, CBDA, CBG, CBN, THCV, apigenin, BCP, and β myrcene constitute at least 98%, at least 99%, or at least 99.9% by weight of the active ingredient(s) of the composition, or any value and range there between. Each possibility represents a separate embodiment of the invention.

In some embodiments, a composition as described herein comprises cannabinoids in a combined concentration ranging from 15-50 µM, 1-40 µM, 5-30 µM, 8-45 µM, 17-55 µM, 10-60 µM, or 20-40 µM. Each possibility represents a separate embodiment of the invention.

In some embodiments, the composition further comprises a pharmaceutically acceptable carrier.

In some embodiments, the composition is formulated for systemic administration. In some embodiments, the composition is formulated for rectal administration. In some embodiments, the composition is formulated for vaginal administration. In some embodiments, the composition is formulated for abdominal administration. In some embodiments, the composition is formulated for subcutaneous administration. In some embodiments, the composition is formulated for intra-peritoneal administration. In some embodiments, the composition is formulated for intravenous administration. In some embodiments, the composition is formulated for administration to a subject.

The term “pharmaceutically acceptable” means suitable for administration to a subject, e.g., a human. For example, the term “pharmaceutically acceptable” can mean approved by a regulatory agency of the Federal or a state government or listed in the U. S. Pharmacopeia or other generally recognized pharmacopeia for use in animals, and more particularly in humans. The term “carrier” refers to a diluent, adjuvant, excipient, or vehicle with which the therapeutic compound is administered. Such pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerin, propylene glycol or other synthetic solvents. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; and agents for the adjustment of tonicity such as sodium chloride or dextrose are also envisioned.

The route of administration of the composition will depend on the disease or condition to be treated. Suitable routes of administration include, but are not limited to, parenteral injections, e.g., intradermal, intravenous, intramuscular, intralesional, subcutaneous, intrathecal, and any other mode of injection as known in the art. Although the bioavailability of the active ingredients to be administered by other routes can be lower than when administered via parenteral injection, by using appropriate compositions it is envisaged that it will be possible to administer the compositions of the invention via transdermal, oral, rectal, vaginal, topical, nasal, inhalation and ocular modes of treatment. In addition, it may be desirable to introduce the pharmaceutical composition of the invention by any suitable route, including intraventricular and intrathecal injection; intraventricular injection may be facilitated by an intraventricular catheter, for example, attached to a reservoir. Pulmonary administration can also be employed, e.g., by use of an inhaler or nebulizer.

In some embodiments, the composition is formulated for systemic administration. In some embodiments, the composition is formulated for abdominal administration. In some embodiments, the composition is formulated for subcutaneous administration. In some embodiments, the composition is formulated for intra-peritoneal administration. In some embodiments, the composition is formulated for intravenous administration. In some embodiments, the composition is formulated for administration to a subject.

In some embodiments, the composition is a pharmaceutical composition.

In some embodiments, the composition is for use in the treatment of a In some embodiments, the composition is for use in the treatment of an inflammatory disease. In some embodiments, the inflammatory disease is characterized by or comprises a high level of IL-6 expression and/or secretion. In some embodiments, the composition is for use in the treatment of an IL-6-related disease, COX-related disease, or both, in a subject in need thereof.

### Methods of Use

In some embodiments, there is provided a method for reducing or inhibiting IL-6 secretion by an immune cell, comprising contacting the immune cell with an effective amount of the composition of the invention, thereby reducing or inhibiting IL-6 secretion by the immune cell.

As used herein, the term “immune cell” refers to any cell of the host defense system within an organism which protects against disease, pathogens, other pathological agents or abnormalities, or divergence from homeostasis.

In some embodiments, the immune cell is obtained or derived from a subject.

As used herein, the term “subject” refers to any subject, particularly a mammalian subject, for whom therapy is desired, for example, a human.

In some embodiments, the immune cell is selected from: a monocyte a macrophage, a lymphocyte, or a mast cell.

In some embodiments, the lymphocyte is a B lymphocyte, a T lymphocyte, or both.

In some embodiments, contacting is contacting in vivo, ex-vivo or in vitro. In some embodiments, ex-vivo or in vitro comprises or is in a test tube or in a plate.

Methods for determining cytokine secretion levels, e.g., IL-6, are common and would be apparent to one of ordinary skill in the art. Non-limiting examples for methods of determining IL-6 secretion levels, include, but are not limited to, immunoassays, such as enzyme-linked immunosorbent assay (ELISA), western blot, dot-blot, MS-MS, or others.

In some embodiments, there is provided a method for treating a subject afflicted with an IL-6 related disease, a COX-related disease, or both, comprising administering to the subject a therapeutically effective amount of the composition of the invention, thereby treating an IL-6 related disease, a COX-related disease, or both, in the subject.

In some embodiments, the method further comprises a step of determining secreted IL-6 level, COX expression level, or both, wherein any one of: (a) a secretion level of IL-6, (b) an expression level of COX, or a combination of (a) and (b), above a predetermined threshold indicates the subject is suitable for treatment with the composition of the invention.

In some embodiments, the determining step is performed before the administering step.

In some embodiments, there is provided a method for selecting a subject suitable for treatment with the composition of the invention, comprising the steps of: (a) determining the secreted IL-6 level, COX expression level, or both, wherein any one of: (i) a secretion level of IL-6, (ii) an expression level of COX, or a combination of (i) and (ii), above a predetermined threshold indicates the subject is suitable for treatment with the composition of the invention, and (b) administering to a subject determined to be suitable for treatment according to step (a) a therapeutically effective amount of the composition of the invention.

In some embodiments, the determining step is performed in the subject or in a sample derived or obtained from the subject. In some embodiments, the sample comprises any bodily fluid, cell, tissue, biopsy, organ, or a combination thereof, derived or obtained from the subject. In some embodiments, the determining step is performed in vivo, ex vivo, or in vitro. In some embodiments, ex vivo or in vitro comprises or is in a test tube or in a plate.

In some embodiments, an IL-6-related disease, a COX-related disease, or both, comprises or is an immune disease, including, but not limited to inflammation.

As used herein, the term “IL-6-related disease” refers to any pathology or a disease wherein IL-6 expression, secretion, or both, initiates, propagates, involved, promotes, enhances, triggers, or any combination thereof, a pathological condition, or a disease or a symptom thereof. In some embodiments, IL-6-related disease is any disease or condition involving IL-6 expression, secretion, or both, in the pathogenesis, pathophysiology, or both, of the disease or condition. In some embodiments, the IL-6 related disease is selected from: rheumatoid arthritis, juvenile rheumatoid arthritis, systemic onset juvenile rheumatoid arthritis, psoriatic arthritis, ankylosing spondylitis, gastric ulcer, seronegative arthropathies, osteoarthritis, inflammatory bowel disease, ulcerative colitis, systemic lupus erythematosus, antiphospholipid syndrome, iridocyclitis/uveitis/optic neuritis, idiopathic pulmonary fibrosis, systemic vasculitis/Wegener’s granulomatosis, sarcoidosis, orchitis/vasectomy reversal procedures, allergic/atopic diseases, asthma, allergic rhinitis, eczema, allergic contact dermatitis, allergic conjunctivitis, hypersensitivity pneumonitis, transplants, organ transplant rejection, graft-versus-host disease, systemic inflammatory response syndrome, sepsis syndrome, gram positive sepsis, gram negative sepsis, culture negative sepsis, fungal sepsis, neutropenic fever, urosepsis, meningococcemia, trauma/hemorrhage, bums, ionizing radiation exposure, acute pancreatitis, adult respiratory distress syndrome, rheumatoid arthritis, alcohol-induced hepatitis, chronic inflammatory pathologies, sarcoidosis, Crohn’s pathology, sickle cell anemia, diabetes, nephrosis, atopic diseases, hypersensitivity reactions, allergic rhinitis, hay fever, perennial rhinitis, conjunctivitis, endometriosis, asthma, urticaria, systemic anaphylaxis, dermatitis, pernicious anemia, hemolytic disease, thrombocytopenia, graft rejection of any organ or tissue, kidney transplant rejection, heart transplant rejection, liver transplant rejection, pancreas transplant rejection, lung transplant rejection, bone marrow transplant (BMT) rejection, skin allograft rejection, cartilage transplant rejection, bone graft rejection, small bowel transplant rejection, fetal thymus implant rejection, parathyroid transplant rejection, xenograft rejection of any organ or tissue, allograft rejection, anti-receptor hypersensitivity reactions, Grave’s disease, Raynaud’s disease, type B insulin-resistant diabetes, asthma, myasthenia gravis, antibody meditated cytotoxicity, type III hypersensitivity reactions, systemic lupus erythematosus, POEMS syndrome (polyneuropathy, organomegaly, endocrinopathy, monoclonal gammopathy, and skin changes syndrome), polyneuropathy, organomegaly, endocrinopathy, monoclonal gammopathy, skin changes syndrome, antiphospholipid syndrome, pemphigus, scleroderma, mixed connective tissue disease, idiopathic Addison’s disease, diabetes mellitus, chronic active hepatitis, primary biliary cirrhosis, vitiligo, vasculitis, post-M cardiotomy syndrome, type IV hypersensitivity, contact dermatitis, hypersensitivity pneumonitis, allograft rejection, granulomas due to intracellular organisms, drug sensitivity, metabolic/idiopathic, Wilson’s disease, hemochromatosis, alpha-1-antitrypsin deficiency, diabetic retinopathy, Hashimoto’s thyroiditis, osteoporosis, hypothalamic-pituitary-adrenal axis evaluation, primary biliary cirrhosis, thyroiditis, encephalomyelitis, cachexia, cystic fibrosis, neonatal chronic lung disease, chronic obstructive pulmonary disease (COPD), familial hematophagocytic lymphohistiocytosis, dermatologic conditions, psoriasis, alopecia, nephrotic syndrome, nephritis, glomerular - nephritis, acute renal failure, hemodialysis, uremia, toxicity, preeclampsia, okt3 therapy, anti-cd3 therapy, cytokine therapy, chemotherapy, radiation therapy (e.g., including but not limited to asthenia, anemia, cachexia, and the like), chronic salicylate intoxication, sleep apnea, obesity, heart failure, sinusitis, and inflammatory bowel disease.

As used herein, the term “COX-related disease” refers to any case wherein COX expression initiates, propagates, is involved, promotes, enhances, triggers, or any combination thereof, a pathological condition, or a disease or a symptom thereof. In some embodiments, COX-related disease is any disease or condition involving COX expression in the pathogenesis, pathophysiology, or both, of the disease or condition. In some embodiments, COX is COX1, COX2, or both. In some embodiments, the COX-related disease is selected from: rheumatoid arthritis, spondyloarthropathies, gouty arthritis, osteoarthritis, systemic lupus erythematosus, juvenile arthritis, asthma, bronchitis, menstrual cramps, tendinitis, bursitis, psoriasis, eczema, burns, dermatitis, inflammatory bowel disease, Crohn’s disease, gastritis, irritable bowel syndrome, ulcerative colitis, cancer, such as colorectal cancer, vascular disease, migraine headaches, periarteritis nodosa, thyroiditis, aplastic anemia, Hodgkin’s disease, sclerodoma, rheumatic fever, type I diabetes, myasthenia gravis, multiple sclerosis, sarcoidosis, nephrotic syndrome, Behcet’s syndrome, polymyositis, gingivitis, hypersensitivity, swelling occurring after injury, myocardial ischemia, retinitis, retinopathies, conjunctivitis, uveitis, ocular photophobia, acute injury to the eye tissue, pulmonary inflammation, such as that associated with viral infections and cystic fibrosis, cortical dementias including Alzheimer’s disease, allergic rhinitis, respiratory distress syndrome, endotoxin shock syndrome, atherosclerosis and central nervous system damage resulting from stroke, ischemia and trauma.

In some embodiments, treating comprises one or more of: increasing immune cell viability, reducing nitric oxide production, reducing COX expression, reducing cytokine secretion, or any combination thereof, in the subject.

In some embodiments, “reduce” or “reducing” is at least a: 10%, 15%, 20%, 25%, 30%, 35%, 40%, 45%, 50%, 55%, 60%, 65%, 70%, 75%, 80%, 85%, 90%, 95%, 97%, 99%, or 100% reduction, or ant value and range therebetween. Each possibility represents a separate embodiment of the invention.

In some embodiments, “increase” or “increasing” is at least a: 10%, 20%, 25%, 30%, 40%, 50%, 60%, 70%, 75%, 80%, 90%, 100%, 150%, 200%, 250%, 300%, 350%, 400%, 450%, 500%, 600%, 700%, 800%, 900%, or 1,000% increase, or any value and range therebetween. Each possibility represents a separate embodiment of the invention.

The terms “reduce”, “reducing”, “inhibit” and “inhibiting” are used interchangeably.

Methods for determining immune cell viability, nitric oxide production, COX expression, and IL-6 secretion level, are common and would be apparent to one of ordinary skill in the art. Non-limiting examples for such methods, include, but are not limited to, immunoassays, such as enzyme-linked immunosorbent assay (ELISA), western blot, dot-blot, MS-MS, RT-PCR, TUNEL apoptosis assay, trypan blue stain, acridine orange/ethidium bromide, real-time RT-PCR, or others, some of which are disclosed hereinbelow.

In some embodiments, the disease is selected from: an inflammatory disease, endometriosis, dysmenorrhea, or dyspareunia. In some embodiments, an inflammatory disease comprises or is an inflammatory lung disease or a viral-induced inflammatory disease.

In some embodiments, the disease is a women’s health related disease or condition.

As used herein, the phrase “women’s health related disease or condition” comprises or consists of any health related disease or condition in women that results from, induced by, or involves inflammation, or any combination thereof.

In some embodiments, a women’s health related disease is induced by inflammation in a woman’s reproductive organ and/or a neighboring tissue thereto. In some embodiments, a neighboring tissue covers or underlies the reproductive organ. In some embodiments, a neighboring tissue comprises a skin tissue, a connective tissue, or both.

In some embodiments a woman’s reproductive organ is selected from: breast, uterus, vulva, vagina, clitoris, ovary, cervix, and fallopian tube.

In some embodiments, a women’s health related disease is induced by inflammation in the endometrium, myometrium, perimetrium, or any combination thereof. In some embodiments, a women’s health related disease is induced by inflammation in the endometrium.

In some embodiments, a women’s health related disease is induced by inflammation in a woman’s urethra.

The phrases “induced by”, “initiated by”, “enhanced by”, “propagated by”, and “involves” are interchangeable.

In some embodiments, a women’s health related disease or condition is selected from: osteoporosis, vaginal atrophy and dryness, hypogonadism, skin atrophy, connective tissue disease, breast, endometrial, ovarian or uterine cancer, hot flashes, physical symptoms of menopause, or any combination thereof.

In some embodiments, administering is topically administering, orally administering, or both.

As used herein, the terms “administering”, “administration”, and like terms refer to any method which, in sound medical practice, delivers a composition containing an active agent to a subject in such a manner as to provide a therapeutic effect. One aspect of the present subject matter provides for topical administration, oral administration, or both, of a therapeutically effective amount of a composition of the present subject matter to a patient in need thereof. Other suitable routes of administration can include parenteral, subcutaneous, intravenous, intramuscular, or intraperitoneal.

The term “therapeutically effective amount” refers to an amount of a drug effective to treat a disease or disorder in a mammal. The term “a therapeutically effective amount” refers to an amount effective, at dosages and for periods of time necessary, to achieve the desired therapeutic or prophylactic result. The exact dosage form and regimen would be determined by the physician according to the patient’s condition.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject’s quality of life.

As used herein, the term “prevention” of a disease, disorder, or condition encompasses the delay, prevention, suppression, or inhibition of the onset of a disease, disorder, or condition. As used in accordance with the presently described subject matter, the term “prevention” relates to a process of prophylaxis in which a subject is exposed to the presently described compositions or composition prior to the induction or onset of the disease/disorder process. This could be done where an individual has a genetic pedigree indicating a predisposition toward occurrence of the disease/disorder to be prevented. For example, this might be true of an individual whose ancestors show a predisposition toward certain types of, for example, inflammatory disorders. The term “suppression” is used to describe a condition wherein the disease/disorder process has already begun but obvious symptoms of the condition have yet to be realized. Thus, the cells of an individual may have the disease/disorder, but no outside signs of the disease/disorder have yet been clinically recognized. In either case, the term prophylaxis can be applied to encompass both prevention and suppression. Conversely, the term “treatment” refers to the clinical application of active agents to combat an already existing condition whose clinical presentation has already been realized in a patient.

All scientific and technical terms used herein have meanings commonly used in the art unless otherwise specified. The definitions provided herein are to facilitate understanding of certain terms used frequently herein and are not meant to limit the scope of the present disclosure.

Before specific aspects and embodiments of the invention are described in detail, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise”, “include” and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Unless specifically stated or obvious from context, as used herein, the term “or” is understood to be inclusive.

Throughout this specification and claims, the word “comprise”, or variations such as “comprises” or “comprising,” indicate the inclusion of any recited integer or group of integers but not the exclusion of any other integer or group of integers.

As used herein, the term “consists essentially of”, or variations such as “consist essentially of” or “consisting essentially of” as used throughout the specification and claims, indicate the inclusion of any recited integer or group of integers, and the optional inclusion of any recited integer or group of integers that do not materially change the basic or novel properties of the specified method, structure or composition.

As used herein, the terms “comprises”, “comprising”, “containing”, “having” and the like can mean “includes”, “including”, and the like; “consisting essentially of or “consists essentially” likewise has the meaning ascribed in U.S. pat. law and the term is open-ended, allowing for the presence of more than that which is recited so long as basic or novel characteristics of that which is recited is not changed by the presence of more than that which is recited, but excludes prior art embodiments. In one embodiment, the terms “comprises”, “comprising”, “having” are/is interchangeable with “consisting”.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Generally, the nomenclature used herein, and the laboratory procedures utilized in the present invention include chemical, molecular, biochemical, and cell biology techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); “Cell Biology: A Laboratory Handbook”, Volumes I-III Cellis, J. E., ed. (1994); The Organic Chemistry of Biological Pathways by John McMurry and Tadhg Begley (Roberts and Company, 2005); Organic Chemistry of Enzyme-Catalyzed Reactions by Richard Silverman (Academic Press, 2002); Organic Chemistry (6th Edition) by Leroy “Skip” G Wade; Organic Chemistry by T. W. Graham Solomons and, Craig Fryhle.

### Materials and Methods

### Cell Culture and Treatment

Cell lines were cultured according to standard mammalian tissue culture protocols and sterile technique. Adherent human epithelial endometriosis 12Z and mouse monocytes RAW 264.7 cell lines were cultured in high glucose Dulbecco’s Modified Eagle Medium (DMEM) supplemented with 1% sodium pyruvate. Human monocytes THP-1 cell line were cultured in RPMI medium 1640. All media was supplemented with 10% fetal bovine serum, streptomycin (100 mg/ml), penicillin (100 U/ml) and Nystatin (12.5 U/ml). Cells were incubated in 5% CO₂ at 37° C. All tissue culture cells were maintained in 75 cm² cell culture treated flask (Eppendorf) and all the media and supplements were obtained from Biological Industries. Treatment were performed by plating cells in a 96 micro well delta surface plates (Eppendorf) in a starting confluence of 1 × 10⁴ cells/well.

### MTT Viability Assay

The viability of the cells following treatment was determined using a commercially available MTT assay kit (Abcam, ab 146345) and performed according to manufacturer’s instructions. Briefly, cells were seeded in a 96-well plate at a density of 1 × 10⁴ cells/well (n=4) in 100 µl cell specific media. After overnight plating, cells were exposed to varying concentrations of cannabis samples in 100 µl of specific media. Then, plates were incubated in a humidified atmosphere containing 5% CO₂ in air at 37° C. for 24 hours. According to the MTT standard protocol, after 24 h treatment, the media was removed, and all cells were incubated with serum-free media containing 0.5 mg/ml MTT for 4 hours at the incubator. The MTT purple crystals formed by the viable cells were dissolved using isopropanol containing 0.04 mol/L HCl. The quantification was determined by measuring the optical density at 570 nm in a spectrophotometer reader (Spark, Tecan). Results were presented as proportional viability (%) by comparing between treated and untreated groups. Cytotoxic potential of an extract was determined according to the following criteria: Non-cytotoxic -Viability is ≥ 70% of vehicle control; Cytotoxic - Viability is reduced to less than 70% of vehicle control.

### Nitric Oxide Detection Assay

RAW 264.7 cells were seeded in a 96-well plate at a density of 1 × 10⁴ cells/well (n=4) in 100 µl cell specific media (DMEM high glucose supplemented with 10% FBS, 1% PSN and 1% Sodium pyruvate). After 24 hours, cells were treated with varying concentrations of cannabis samples using specific media containing 5% FBS. After 1 hour, treated and untreated cells, were stimulated by Lipopolysaccharides (LPS) at a final concentration of 1 µg/ml. Cells supernatants were harvest after 24 h for nitric oxide radical (NO•) assay by addition of 50 µl of the supernatant to an equal volume of Griess reagent (1% sulfanilamide, 0.1% naphthalene diamine and 2% H₃PO₄). After 10 min of incubation, the resultant color was measured at 540 nm. Results were presented as proportional (%) by comparing between treated and untreated groups. Anti-inflammatory potential of an extract in preventing Nitric oxide production was determined by the ratio between nitric oxide and cell viability values: Ratio < 0.5 = High effect; Ratio between 0.5-0.7 = Moderate effect; Ratio > 0.8 = Low effect; and Ratio > 1 = No effect.

### Protein Extraction and Western Blot Analysis

Whole cell lysate was prepared by washing cells pellets with 1 × Phosphate buffer saline (Biological Industries), resuspending it in ice-cold T lysis buffer [50 mM Tris-Cl (pH 7.5), 150 mM NaCl, 1 mM EDTA, 1% Triton × and 1 × halt™ protease and phosphatase inhibitor cocktail] and incubating for 30 minutes in ice. The lysate was followed by centrifugation at 13,800 g for 10 min at 4° C. to clear the cellular debris. Total protein was quantified using the BCA protein assay kit (Thermo Scientific). Equal amount of protein was resolved on precast Bolt™ 4-12% Bis-Tris Plus polyacrylamide gel (Invitrogen), electrotransferred to precast nitrocellulose stacks using iBlot®2 system (Invitrogen) and western blot analysis was performed using the antibodies described above. Immunodetection was performed by blocking the membranes for 1 h in TNT buffer [10 mM Tris-Cl (pH 7.5), 150 mM NaCl, 0.05% Tween-20] containing 5% powdered non-fat milk followed by addition of the primary antibody (as indicated) in TNT for 2 h at room temperature. Specifically bound primary antibodies were detected with peroxidase-coupled secondary antibodies and developed by enhanced chemiluminescence (biological industries) according to manufacturer’s instructions and quantitated using Azure biosystem C280.

### Immunoblot Analysis

Immunoblot analysis was performed using antibodies against GAPDH (1:5,000 dilution; ab181602, Abcam), Cox-1 (1:1,000 dilution; ab109025, Abcam), Cox-2 (1:1,000 dilution; ab62331, Abcam). Species-specific HRP-labeled secondary antibodies were then added. The blots were visualized using enhanced chemiluminescence (biological industries) and quantitated using Azure biosystem C280.

### Agarose Spot

0.1 g of low-melting point agarose (Sigma-Aldrich) was placed into a 100-mL beaker and diluted into 20 mL PBS to make a 0.5% agarose solution. This was heated on a hot plate in the cell culture hood until boiling, swirled to facilitate complete dissolution, and then taken off of the heat. When the temperature cooled to 40° C., 90 µL of agarose solution was pipetted into a 1.5-mL Eppendorf tube containing 10 µL of cannabis sample. Ten-microliter spots of agarose were pipetted, using cut pipet, as rapidly as possible onto 35-mm glass-bottomed dishes (Cat. no. BN200350; Bar Naor), and allowed to cool for ~5 min at 4° C. At this point cells were plated into spot-containing dishes in the presence of 10% FCS cell culture media and allowed to adhere for 4 h. Cells were then transferred into cell culture with 5% FCS, replaced into the incubator overnight and analyzed by microscopy the next morning. Imaging was performed on a Nikon TE300 inverted microscope with a 10× objective (Nikon, Kingston upon Thames, Surrey, UK), and for each spot we recorded the field that contained the highest apparent number of motile cells penetrating furthest underneath the agarose spot.

### Homogeneous Time-resolvedfluorescence (HTRF)

THP-1 monocytic cells were seeded in a 96-well plate (Eppendorf) at a density of 3 × 10⁵ cells/well (n=4) in 100 µl cell specific media. For differentiation, phorbol-12-myristate 13-acetate (PMA) (Sigma-Aldrich) was added to a final concentration of 200 nM. After 3 days, the PMA supplemented media was removed, cells were washed 2 times with PBS and rested in fresh PMA-free media for further 24 hours in order to obtain phenotypic characteristics of macrophages (Daigneault et al., 2010). After 24 hours, cells were treated with varying concentrations of cannabis samples using specific media containing 5% FBS. After 1 hour, treated and untreated cells, were stimulated by Lipopolysaccharides (LPS) at a final concentration of 1 µg/ml.

Cells supernatants were harvest after 24 h and diluted up to 5 times in working media to avoid the hook effect. HTRF assays were performed in white 96-well plate (CisBio Bioassays) with a total working volume of 20 µL. All HTRF reagents were purchased from CisBio Bioassays and reconstituted according to the supplier protocols. For each assay 16 µL of diluted supernatants samples were incubate with 4 µL mixed solution containing donor and acceptor antibodies.

After 2 h incubation HTRF signals were measured using SPARK® multimode microplate reader with an excitation filter at 320 nm and fluorescence wavelength measurement at 620 and 665 nm, an integration delay of 60 µs and an integration time of 400 µs. Results were analyzed with a two-wavelengths signal ratio: [intensity (665 nm)/intensity (620 nm)]×10⁴. Calculation and analysis of cytokines release was performed according to the supplier protocols and using the 4PL 1/Y2 formula in GraphPad Prism software.

### Example 1

### The Effects of Specific Cannabinoids on Cellular Parameters

The inventors examined the effects of isolated cannabinoids and their combinations on various cellular parameters, including cell viability and nitric oxide production, in vitro.

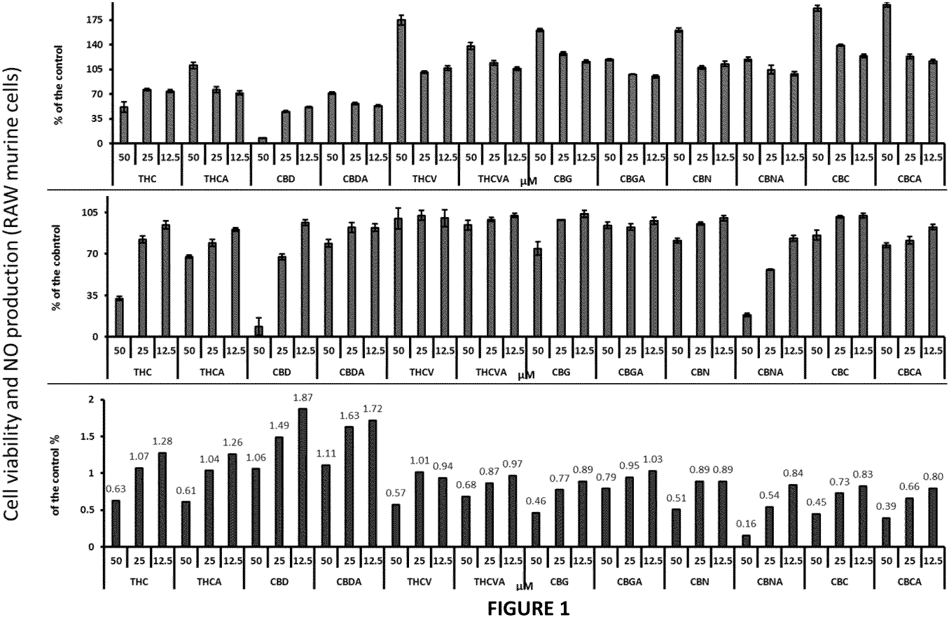

The inventors showed that 50 µM of any one of: THCA, THCV, THCVA, CBG, CBN, CBNA, CBC, and CBCA, reduced nitric oxide production while minimally affecting cell viability or not affecting cell viability at all (FIG. 1). CBNA and CBCA also showed a comparable effect when applied at lower dose, 25 µM.

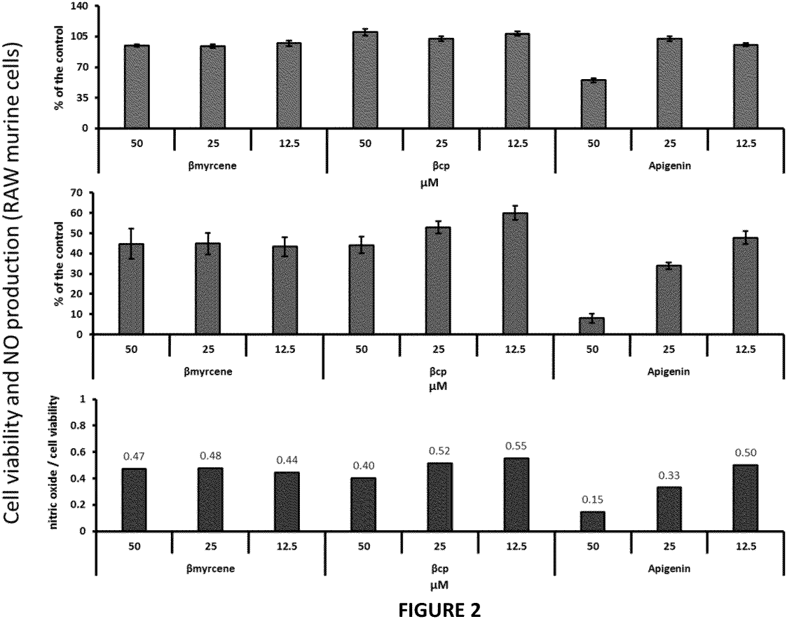

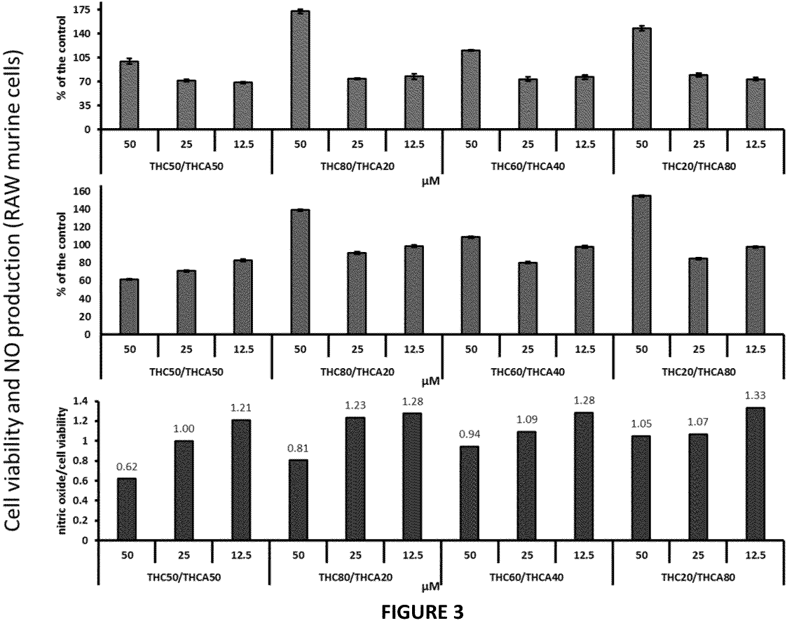

Further, the inventors examined the aforementioned cellular parameters, when the cells were treated with different combinations of cannabinoids and their corresponding acid precursor. The inventors showed that combination of THC and THCA, at a concentration of 50 µM, 25 µM, or 12.5 µM, wherein the weight per weight ratio of THC to THCA is 1:1, markedly reduced nitric oxide production, while minimally affecting cell viability or not affecting cell viability at all (FIG. 3). When terpenes and/or flavonoids were applied separately (e.g., without cannabinoids), nitric oxide production was found to be significantly reduced with no substantial effect on cell viability (excluding 50 µM of Apigenin; FIG. 2).

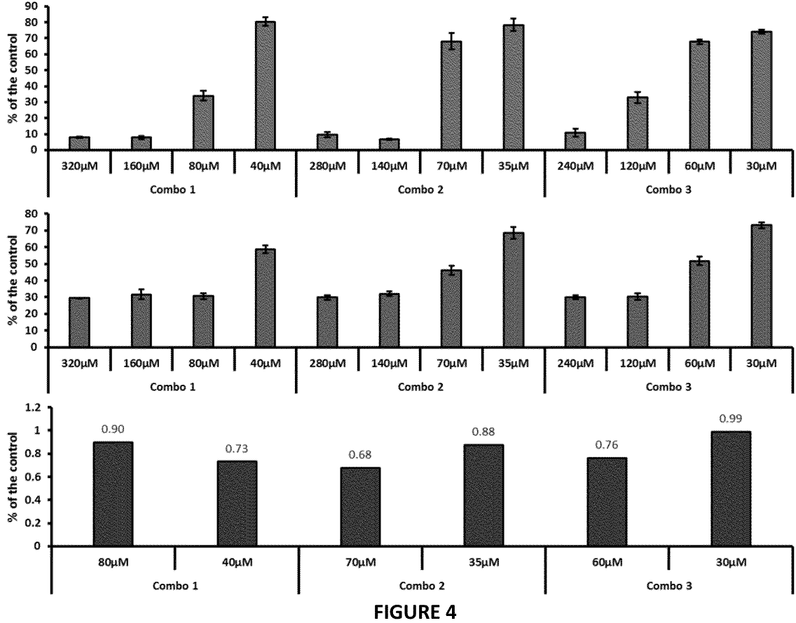

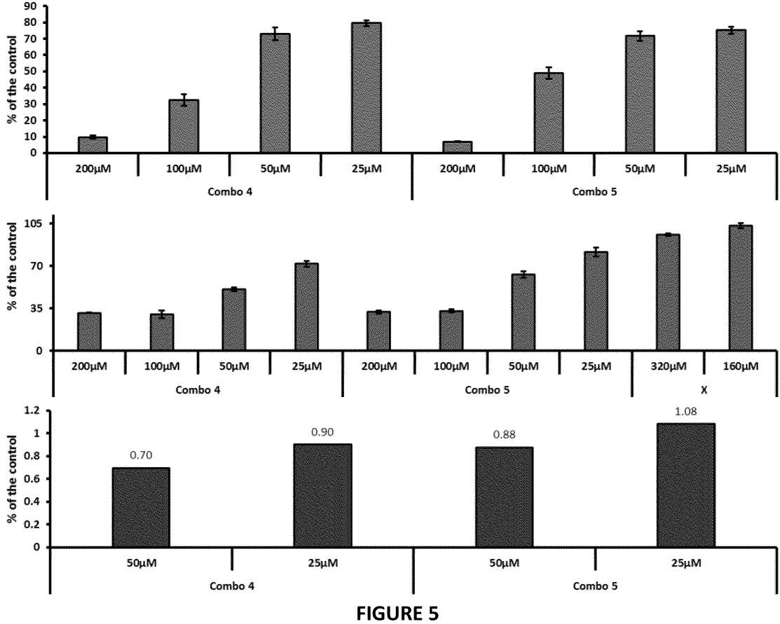

The inventors showed that combinations of cannabinoids, their corresponding acid precursors, which further comprised terpenes and/or flavonoids, also substantially reduced nitric oxide production while minimally affecting cell viability or not affecting cell viability at all (FIGS. 4-5), e.g., 70 µM of combination 2, and 60 µM of combination 3, to name a few.

### Example 2

### The Effects of Specific Cannabinoids on Cyclooxygenase Expression and IL-6 Secretion

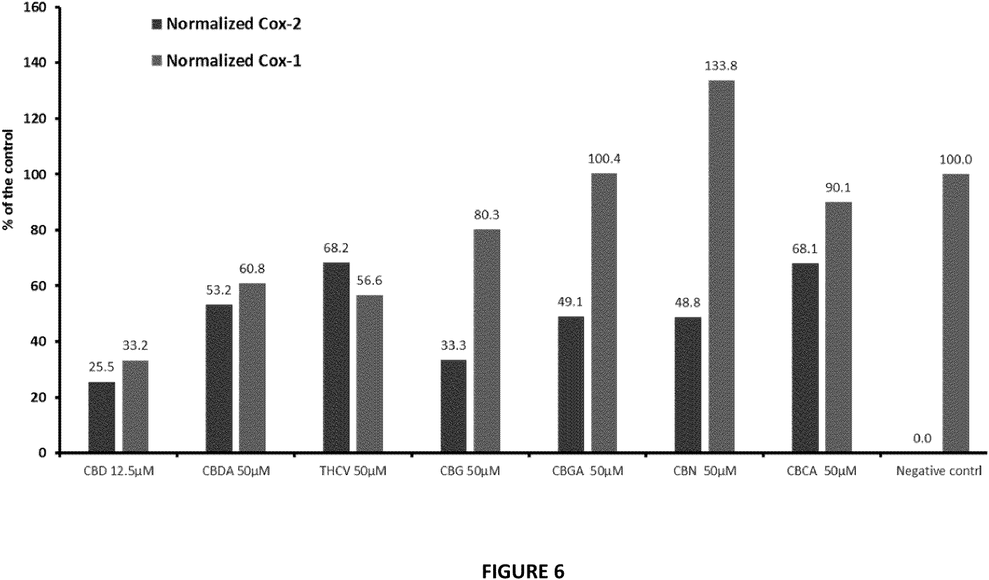

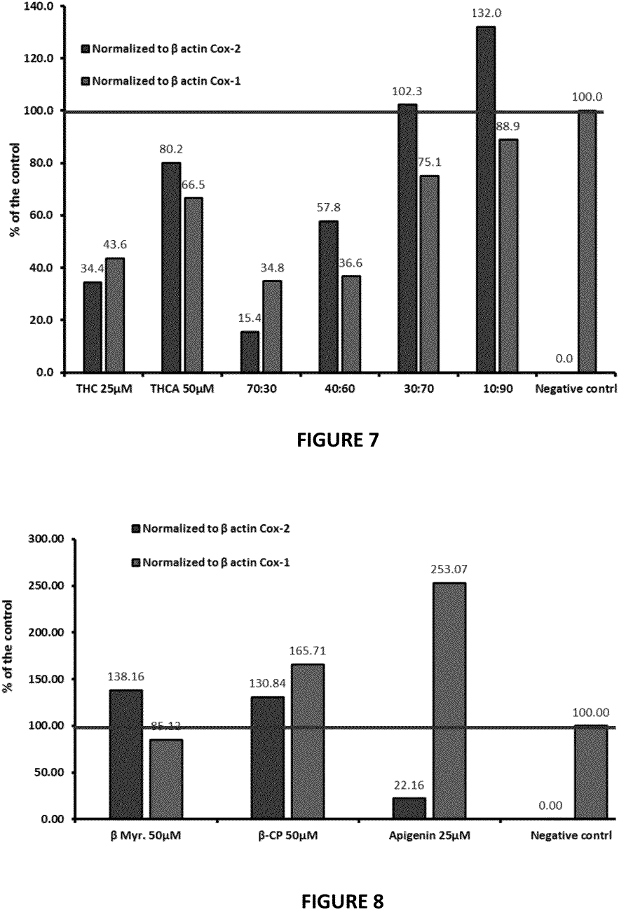

The inventors examined the effect of each of the abovementioned cannabinoids, terpenes and/or flavonoids, on the expression of cyclooxygenase 1 and 2 (FIGS. 6-8).

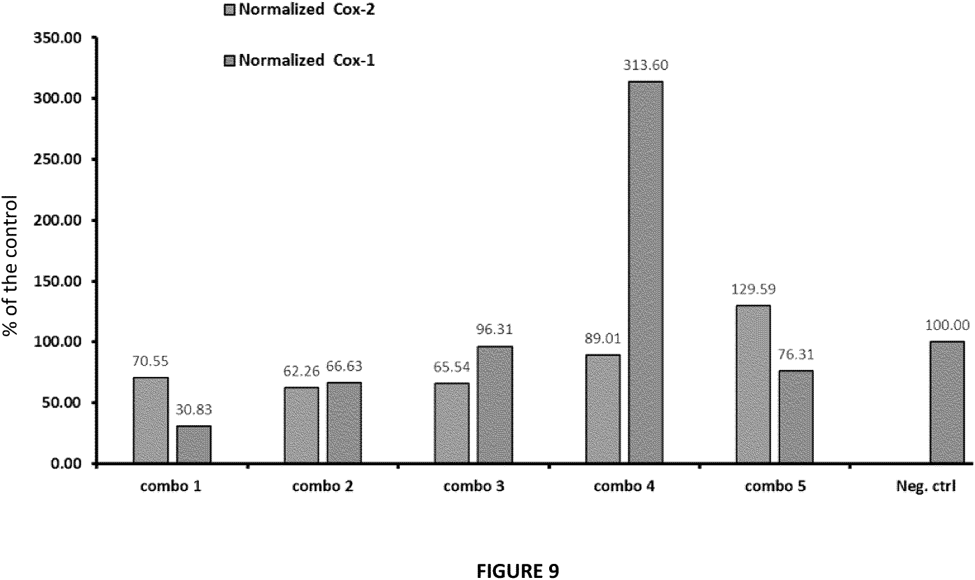

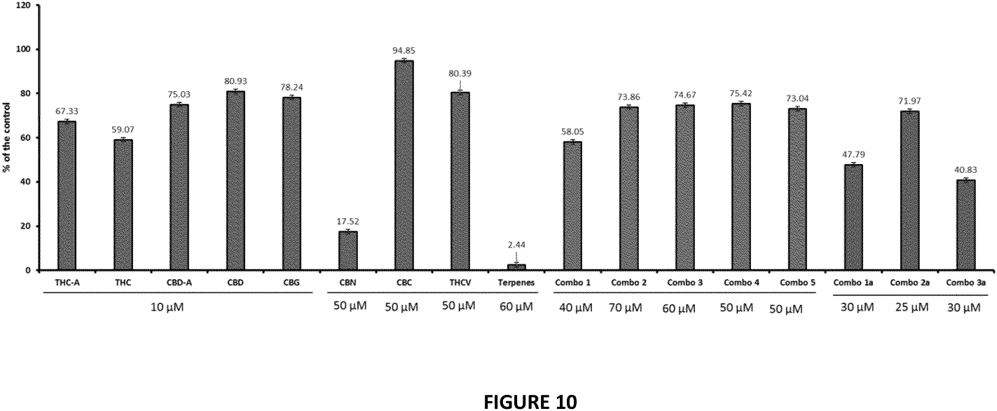

Further, the inventors showed that the expression levels of COX-1 in cells treated with combination 2 or combination 3 (as in Example 1) did not exceed those of the negative control, whereas COX-2 expression were lower than in any of the other tested combinations (FIG. 9). Further, both combination 2 and combination 3 reduced interleukin-6 secretion by ~25% compared to control (FIG. 10).

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1. A composition comprising: cannabidiol (CBD), cannabidiolic acid (CBDA), tetrahydrocannabinol (THC), and tetrahydrocannabinol acid (THCA), wherein:
i. the weight per weight or molar ratio of CBD to CBDA is in the range of 1:2 to 2:1,
ii. the w/w (or molar) ratio of THC to THCA is in the range of 1:1 to 4:1, and
iii. the w/w (or molar) ratio of CBD and CBDA to THC and THCA is in the range of 1:2 to 2:1.

2. The composition of claim 1, wherein the w/w or molar ratio of THC to THCA is in the range of 2:1 to 2.5:1.

3. The composition of claim 1, wherein the w/w or molar ratio of CBD to CBDA is 1:1.

4. The composition of claim 1, wherein the w/w or molar ratio of CBD and CBDA to THC and THCA is 1:1.

5. The composition of claim 1, further comprising an additional compound selected from the group consisting of: cannabigerol (CBG), cannabinol (CBN), tetrahydrocannabivarin (THCV), apigenin, beta caryophyllene (BCP), β myrcene, and any combination thereof.

6. The composition of claim 5, wherein the w/w or molar ratio of: (a) the combination of CBD and CBDA, (b) the combination of THC and THCA, or (c) the combination of (a) and (b), to said additional compound is in the range of 1:1 to 1:6.

7. A composition consisting essentially of CBD, CBDA, THC, THCA, CBG, apigenin, BCP, and β myrcene.

8. The composition of claim 7, wherein the molar ratio of: (a) the combination of CBD and CBDA, (b) the combination of THC and THCA, or (c) the combination of (a) and (b) to any one of: CBG, apigenin, BCP, and β myrcene, is in the range of 1:1 to 1:10.

9. (canceled)

10. (canceled)

11. (canceled)

12. (canceled)

13. A composition consisting essentially of CBD, CBDA, CBG, CBN, THCV, apigenin, BCP, and β myrcene.

14. The composition of claim 13, wherein the molar ratio of the combination of CBD and CBDA to any one of: CBG, CBN, THCV, apigenin, BCP, and β myrcene, is in the range of 1:1 to 1:10.

15. The composition of claim 1, comprising a total concentration of cannabinoids ranging from 15 to 50 µM.

16. The composition of claim 1, further comprising a pharmaceutically acceptable carrier.

17. (canceled)

18. (canceled)

19. (canceled)

20. A method for reducing or inhibiting IL-6 secretion by an immune cell, comprising contacting said immune cell with an effective amount of the composition of claim 1, thereby reducing or inhibiting IL-6 secretion by the immune cell.

21. The method of claim 20, wherein said immune cell comprises a monocyte, a macrophage, or a combination thereof.

22. The method of claim 20, wherein said contacting is contacting in-vivo, ex-vivo or in-vitro.

23. A method for treating a subject afflicted with an IL-6-related disease, COX-related disease, or both, comprising the step of administering to said subject a therapeutically effective amount of the composition of claim 1, thereby treating the subject afflicted with an IL-6-related disease, COX-related disease, or both.

24. The method of claim 23, further comprising a step of determining the level of secreted IL-6, COX expression level, or both, of said subject, wherein any one of: (a) a secretion level of IL-6, (b) an expression level of COX , or a combination of (a) and (b), above a predetermined threshold indicates the subject is suitable for treatment with said composition, and wherein said determining step is performed before said administering step.

25. The method of claim 23, wherein said disease is selected from the group consisting of: an immune disease, an inflammatory disease, endometriosis, dysmenorrhea, and dyspareunia, and optionally wherein said inflammatory disease is an inflammatory lung disease, or a viral induced inflammation.

26. (canceled)

27. The method of claim 23, wherein said administering is topically administering, orally administering, or both.

28. The method of claim 23, wherein said treating comprises one or more of: increasing immune cell viability, reducing nitric oxide production, reducing COX expression, reducing IL-6 secretion, or any combination thereof, in said subject, optionally wherein said COX is COX1. COX2, or both.

29. (canceled)
